(19) United States
(12) Patent Application Publication
(10) Pub. No.: US 2023/0338647 A1
(43) Pub. Date: Oct. 26, 2023

(54) SUBCUTANEOUSLY IMPLANTED DEVICE FOR GASTROINTESTINAL ADMINISTRATION OF SUBSTANCES

(71) Applicant: Endopump Ltd., Yokneam (IL)

(72) Inventors: Eliahu Heldman, Rehovot (IL); Moshe Kushnir, Ramat Gan (IL); Tal Youdim, Kiryat Tivon (IL)

(73) Assignee: Endopump Ltd., Yokneam (IL)

(21) Appl. No.: 17/792,764

(22) PCT Filed: Dec. 17, 2019
(86) PCT No.: PCT/IB2019/060921

§ 371 Date: Jul. 14, 2022

Related U.S. Application Data
(60) Provisional application No. 62/780,408, filed on Dec. 17, 2018.

(51) Int. Cl. A61K 31/198; A61M 5/142; A61K 31/15; A61M 5/168; A61K 9/08; A61M 5/172

(52) U.S. Cl. CPC A61M 5/14276; A61K 9/08; A61K 31/15; A61K 31/198; A61M 5/16804; A61M 5/172; A61M 2205/04; A61M 2205/3507; A61M 2205/8206; A61M 2210/1057

(57) ABSTRACT

A method includes using a subcutaneously-implanted pump to deliver to a portion of a gastrointestinal tract a formulation including a levodopa compound and a carbidopa compound which provides a treatment for a neurological disease, such as Parkinson’s disease.

### FIELD OF THE INVENTION

The present invention generally relates to a subcutaneously implanted device and method for gastrointestinal administration of substances, such as levodopa compound and carbidopa compound together with enteral absorbance enhancer, for treatment of Parkinson’s disease and other diseases that require continuous drug delivery.

### BACKGROUND OF THE INVENTION

The most important symptomatology of Parkinson’s disease (PD) results from dopaminergic neuron degeneration in the substantia nigra. Progressive degeneration and/or death of the dopaminergic neurons often results in problems with movement (e.g., ataxias, rigidity, tremors, etc.).

Progressive degeneration of the dopaminergic pathway in Parkinson’s disease results in reduced level of the neurotransmitter dopamine in the brain, which manifests itself as symptoms of slowness of movement (e.g., bradykinesia), rigidity, tremor and poor balance.

Biochemically, dopamine (3,4-dihydroxyphenethylamine) is formed by metabolism (decarboxylation) of the dopamine precursor - levodopa (L-3,4-dihydroxyphenylalanine; abbreviated as L-dopa or LD) through the enzyme aromatic L-amino acid decarboxylase (also known as DOPA decarboxylase (DDC)), both in the brain and in peripheral tissues. Levodopa is in turn produced from the amino acid L-tyrosine by the enzyme tyrosine hydroxylase (TH). The most common treatment of PD aims at restoring the dopamine level in the brain. This treatment is symptomatic and not therapeutic, but it enables the patient to function almost normally, at least for some years before the emergence of late motor complications. Administration of dopamine itself is ineffective because it does not cross the blood-brain barrier and causes severe adverse effects, such as orthostatic disturbances. However, since the precursor of dopamine, levodopa, does cross the blood-brain barrier, and is converted to dopamine in the brain, administration of levodopa has for a long time been, and still is, the drug of first choice for PD treatment. To prevent the conversion of levodopa to dopamine in the blood circulation and peripheral tissues, the patient receives carbidopa or benserazide, each of these is an inhibitor of DOPA decarboxylase that does not cross the blood-brain barrier (BBB), thus making the levodopa in the blood circulation more available to the brain and at the same time reduces the adverse effects of peripheral dopamine.

Even when L-dopa and carbidopa are co-administered, it is still difficult to consistently maintain continuous therapeutic dopamine levels in the brain due to the relatively short half-life of L-dopa in the blood circulation. In addition, the tolerance of many patients to variability in dopamine levels in the brain decreases as the disease progresses. It has been shown that keeping the blood level of levodopa constant, without the fluctuations that occur due to intermittent oral administration of levodopa/carbidopa preparations, prevents the development of the late motor complications and can even reverse them.

One approach for reducing fluctuations in blood levels of levodopa, and consequently, preventing the deleterious fluctuations in dopamine levels in the brain, is a continuous delivery of an adjustable dose of an L-dopa/carbidopa gel via duodenal administration known by its commercial name, DUODOPA in Europe and DUOPA in the United States. DUODOPA is a suspension of L-dopa/carbidopa monohydrate (the concentration of the L-dopa/carbidopa in the formulation used for Duodopa is about 2.5% and the ratio between L-Dopa to carbidopa is 4:1) in an aqueous gel (carboxymethyl cellulose sodium) having a viscosity that permits homogeneous distribution of micronized substance particles. The gel is delivered to the proximal small intestine through a jejunal tube inserted through a percutaneous endoscopic gastrostomy port. DUODOPA is packaged in medication cassette reservoirs and continuously administered via a software-controlled ambulatory infusion pump.

One of the major disadvantages of DUODOPA is the size of the cassette that contains the levodopa/carbidopa gel and the accompanied pump; together they are clumsy and lead to significant inconvenience to the patient. For example, the patient has to remove the device during shower and the need to carry an external device all the time limits the patient’s daily activities. The need to replace the cassette of the L-dopa/carbidopa formulation daily, is another disadvantage of DUODOPA.

The prior art lacks a device that can provide continuous gastrointestinal administration of levodopa, which is small enough to become “a part of the patient” (such as subcutaneously implanted device) and can hold enough levodopa/carbidopa for several days, in order to be more patient compatible. Such a subcutaneously implanted device bypasses the need to have an open port that may be contaminated and cause inconvenience to the patient. Another advantage of an implanted device is the safety of the catheter connected to the duodenum without the possibility to detach. In contrast, external pumps have a high risk of the catheter detaching itself from the duodenum because of the movement of the patient and the risk of infection on the penetration site. One of the challenges to the development of a smaller device (compared to DUODOPA), which can be subcutaneously implanted and filled in a frequency of less than once a day, or otherwise improved modes of administering L-dopa and carbidopa has been the low solubility of those compounds. Also, the currently available formulations that contain high concentrations of levodopa and carbidopa are not concentrated enough to allow storage in a small volume that could be inserted into a subcutaneously implanted device and that will be enough for a several days supply. Moreover, the currently available formulations that contain high concentrations of levodopa and carbidopa are viscous and are not suitable for administration via a small-size device that requires thin tubing that connects the device to the duodenum. Stable, and highly concentrated, and less viscous formulations comprising L-dopa and carbidopa (or compounds capable of in vivo bioconversion to L-dopa and/or carbidopa) can provide advantages over existing intestinal infusion therapy. Another desired feature of a formulation that contains high concentrations of levodopa and carbidopa is fast absorption in the duodenum and the proximal jejunum to prevent loss of the drugs that are not absorbed well further on in the GI tract. Therefore, addition of intestinal absorbance enhancer to the formulation improves significantly its performance and allows reducing the daily dose.

PCT Patent Application WO 2018/224501, assigned to Dizlin Pharmaceuticals AB, describes an aqueous pharmaceutical solution for use in the treatment of diseases of the central nervous system (CNS), the solution comprising at least 5 mg/ml dissolved levodopa, and having a pH in the range of 3.0 to 8.5. The solution is provided by mixing a) an aqueous stock solution comprising levodopa, said stock solution having a pH of less than 2.8 at 25° C. and b) an aqueous buffering solution, for increasing the pH of the stock solution, the buffering solution having a pH of at least 4.0 at 25° C.

PCT Patent Application WO 2018/154447, assigned to Neuroderm Ltd., describes formulations containing a carbidopa prodrug and/or a levodopa prodrug, and one, two or more antioxidants. One of the delivery methods for the formulation is the use of an insulin pump, which is external to the body, to pump the formulation to subcutaneous tissue.

However, none of the prior art describes using a subcutaneously-implanted pump to deliver levodopa/carbidopa substances. None of the prior art describes a formulation containing levodopa and carbidopa with a viscosity lower than 50 centipoise at room temperature (25° C.) and none of the prior art describes a formulation of levodopa and carbidopa at a concentration of more than 300 mg/ml, which allows several days of drug supply in a volume that suitable for subcutaneously implanted device.

### SUMMARY OF THE INVENTION

The present invention seeks to provide a novel and improved subcutaneously implanted device and method for gastrointestinal administration of substances, as described in detail below. In a preferred embodiment, the substance is a solution containing levodopa and carbidopa for use in the treatment of diseases of the central nervous system, such as but not limited to, Parkinson’s disease.

Other substances that can be used with the device include, without limitation, dopaminergic agonists such as lisuride, mono amine oxidase (MAO) inhibitors such as rasagiline and selegiline, catechol-O-methyl-transferase (COMT) inhibitors such as entacapone, or amantadine.

In contrast to the prior art, the substance has low viscosity (not greater than 50 centipoise) which makes it suitable for a miniature subcutaneous pump and miniature tubing, yet the concentration of the API (active pharmaceutical ingredient) is 30% or greater.

The device is implanted subcutaneously and includes a pump and a reservoir that contains about 30-50 ml formulation. The device is connected to the gastrointestinal (GI) tract for direct delivery into the duodenum or other parts of the GI tract, such as but not limited to, the stomach, pylorus, jejunum or other portions of the small or large intestines. In contrast with prior art devices and methods, the formulation contains high concentrations of levodopa and carbidopa compounds, and yet has sufficiently low viscosity that enables the present device to deliver the formulation over long periods of time without the pump being overworked and without the cannula becoming clogged.

For example, the ratio between levodopa compound and carbidopa compound is generally 4:1, respectively, and the concentration of levodopa compound (e.g. levodopa, or levodopa ester or levodopa salts) is in the range of 300-1000 mg/ml and that of the carbidopa compound (e.g. carbidopa, or carbidopa ester) is in the range of 75-300 mg/ml. The formulation also contains an intestinal absorbance enhancer, such as, but not limited to, cyclodextrins, or chitosan, or dicarboxylic acids, to enhance the absorbance of the active drugs via the GI tract. Benserazide or other dopa decarboxylase inhibitors can be used as an alternative to carbidopa. The viscosity of the formulation that includes levodopa-carbidopa substance is in the range of 1-50 centipoise.

The reservoir is implanted under the skin and can be refilled from outside the body. It can also be washed when necessary before refilling. The subcutaneously implanted device is connected to the duodenum or other part of the GI tract (such as by a cannula implanted in the stomach, or passes through the pylorus to the duodenum or proximal jejunum, or by a needle or cannula that enters directly into the duodenum or proximal jejunum) with an anchoring device and continuously provides the formulation to the GI tract in order to maintain a constant level of at least 1000 ng/ml levodopa in the blood. The formulation contains ingredients that increase absorbance in the duodenum into the blood circulation, emulsifier to stabilize the emulsion and a compatible solvent.

The device is able to manipulate the level of levodopa in the blood and to administer a bolus, either by the patient or remotely by a controller or by a physician/nurse.

There is thus provided in accordance with a non-limiting embodiment of the present invention a method of treating a neurological disease, including using a subcutaneously-implanted pump to deliver to a portion of a gastrointestinal tract a formulation including a levodopa compound and a carbidopa compound which provides a treatment for a neurological disease (e.g., Parkinson’s disease). In one example, a viscosity of the formulation is not greater than 50 centipoise at 30-41° C. (e.g., at 37° C.). In another example, a viscosity of the formulation is in a range of 1-50 centipoise at 25° C.

In accordance with a non-limiting embodiment of the invention the formulation also includes an intestinal absorbance enhancer.

In one example, a ratio between the levodopa compound and the carbidopa compound is in a range of 3.5:1 - 10:1. The levodopa compound may be in a range of 300-1000 mg/ml and the carbidopa compound may be in a range of 75-300 mg/ml.

In one example, the levodopa compound and the carbidopa compound constitute together at least 30% by weight of the formulation. In another example, the levodopa compound and the carbidopa compound constitute together 50% by weight of the formulation.

In accordance with a non-limiting embodiment of the invention a controller is in communication with the pump to control operation of the pump. A sensor may be in communication with the controller, configured to sense if the formulation contacts tissue in a peritoneal cavity outside a duodenum of the gastrointestinal tract, and if so, the controller stops operation of the pump.

There is provided in accordance with a non-limiting embodiment of the invention a method of making a formulation including a levodopa compound and a carbidopa compound, including dissolving a powder including a levodopa compound and a carbidopa compound in acidic water to form a solution, performing sonication, freezing and lyophilization of the solution to form a substance, and performing reconstitution of the substance in a reconstitution matrix to form a formulation, wherein the levodopa compound and the carbidopa compound are at least 30% by weight of the formulation.

In addition to or alternative to the sonication, vortexing of the solution may also be done. The matrix may include water, acidic water with a pH in a range of 1-3, polyethylene glycol or polyvinylpyrrolidone. The levodopa compound may include levodopa, or salts of levodopa, or levodopa ester including ethyl or propyl isopropyl esters, or other levodopa products, and the carbidopa compound may include carbidopa, or carbidopa ester including carbidopa propyl ester or carbidopa isopropyl ester. The formulation may include benserazide or other dopa decarboxylase inhibitor.

### BRIEF DESCRIPTION OF THE DRAWINGS

The present invention will be understood and appreciated more fully from the following detailed description, taken in conjunction with the drawings in which:

FIG. 1 is a simplified illustration of a device that is implanted subcutaneously and which delivers a substance into the gastrointestinal (GI) tract, constructed and operative in accordance with a non-limiting embodiment of the present invention; and

FIG. 2 is a simplified illustration of the device of FIG. 1.

### DETAILED DESCRIPTION OF EMBODIMENTS

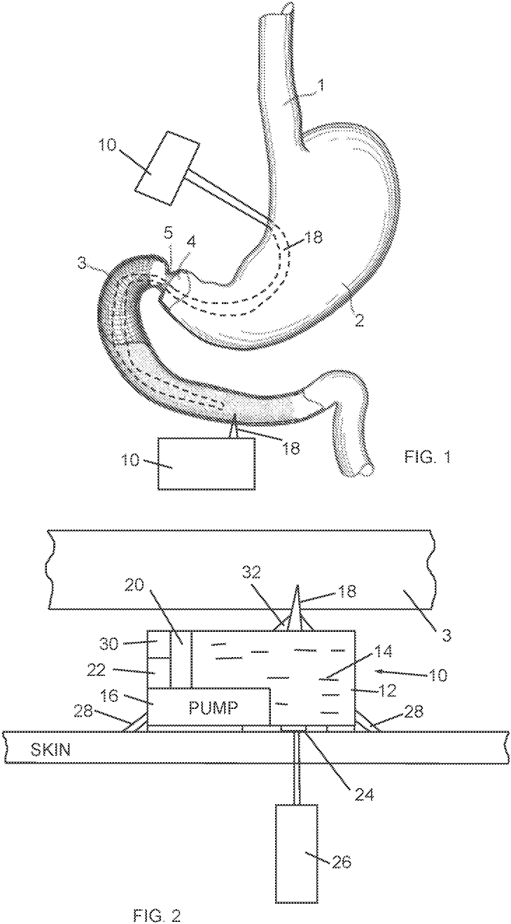

Reference is now made to FIG. 1, which illustrates a device 10, constructed and operative in accordance with a non-limiting embodiment of the present invention, subcutaneously implanted in a patient.

FIG. 1 illustrates a portion of the gastrointestinal (GI) tract, including the esophagus 1, stomach 2, and duodenum 3 (proximal portion of the small intestine). The pyloric part 4 of the stomach leads to the duodenum 3 by way of the pylorus 5.

Reference is now made to FIG. 2. The device 10 includes a reservoir 12 for storing therein a formulation (substance) 14 for delivery to the GI tract 3. The amount of formulation 14 contained in reservoir 12, may be sufficient for continuous delivery of at least 24 hours, or alternatively several days or alternatively other time periods.

The device 10 is implanted under the skin (subcutaneously) and it includes a pump 16 for pumping substance 14 from reservoir 12 via a cannula 18 into the GI tract 3.

In one embodiment, the formulation 14 contains high concentrations of levodopa and carbidopa or levodopa ester and carbidopa ester, or other products of levodopa and carbidopa, together with a compound that enhances the absorption of the levodopa/carbidopa compounds into the GI tract; yet the formulation has sufficiently low viscosity so that the formulation can be delivered over long periods of time with the subcutaneously implanted pump and cannula, without the pump being overworked and without the cannula becoming clogged.

For example, without limitation, the ratio between the levodopa compound and the carbidopa compound is generally 4:1; levodopa compound (e.g. levodopa, or levodopa ester) is in the range of 300-1000 mg/ml and carbidopa compound (carbidopa, or carbidopa ester) is in the range of 75-300 mg/ml. The formulation may also contain an intestinal absorbance enhancer, such as, but not limited to, chitosan, to enhance the absorbance of the active drugs via the GI tract. The viscosity of the formulation that contains all the ingredients, including levodopa-carbidopa substance is in the range of 1-50 centipoise.

Without limitation, in one example, substance 14 may include several levodopa esters, including the ethyl ester, propyl ester, isopropyl ester and butyl ester. In experiments, these esters were successfully dissolved in water to which was added acetic acid until attaining pH 5 (in some cases pH 4.5). The ethyl ester had the highest solubility, but the propyl and the isopropyl esters were also soluble at a concentration of 400 mg/ml. The propyl ester of carbidopa was solubilized under the same conditions and a clear solution of 100 mg/ml of the carbidopa propyl ester was obtained. A mixture of each of the above levodopa esters with carbidopa propyl ester was prepared at a ratio of 4:1 (levodopa ester/carbidopa ester) and a clear solution was obtained. The solution of the mixture was stable for at least two weeks based on chromatographic determination of levodopa esters and carbidopa ester. The formulations of the levodopa esters together with carbidopa ester were prepared in diluted acetic acid and ascorbic acid at a pH 5. Each one of the above mentioned esters have good stability.

Accordingly, levodopa and carbidopa formulations are stabilized using pH 4.5-5. The levodopa and carbidopa esters can be administered into the GI tract over extended periods by pump 16.

Pump 16 may be, without limitation, a diaphragm pump, peristaltic pump, motor-driven pump or any other suitable pump. Pump 16 may be controlled by a controller 20, which may be part of device 10, or alternatively, may be external and communicates wirelessly with pump 16. A battery 22 may be provided in device 10 for powering the pump and controller and any other electrical components of device 10.

The cannula 18 may have a spike or other end for entering the GI tract 3. The point of entry may be at any suitable portion of the GI tract 3, such as but not limited to, the stomach, any portion of the duodenum, or near the proximal part of the jejunum.

Alternatively, as seen in FIG. 1, device 10 may be implanted subcutaneously and cannula 18 may pass through the stomach wall, and then placed in the stomach near the pylorus, or pass through the pylorus to the duodenum, or proximal jejunum.

The device 10 may include a port 24 communicating with reservoir 12 and accessible from outside the skin, such as by a syringe 26. Port 24 may be used to refill reservoir 12 with substance 14. Additionally, port 24 (or another dedicated port) may be used to access cannula 18 for draining and washing cannula 18 (with saline or water, for example).

Device 10 may be anchored to the skin with sutures, barbs and other fixation devices 28, without causing trauma or discomfort to the patient. Device 10 may also include a cannula fixation device 32 for anchoring cannula 18 to the duodenum and for sealing cannula 18 to the duodenum.

Contact between the formulation 14 and tissues in the peritoneal cavity should be avoided. In accordance with a non-limiting embodiment of the present invention, device 10 includes one or more sensors 30 that sense if the formulation 14 contacts tissue in the peritoneal cavity outside the duodenum. Alternatively, sensor 30 may sense the flow of the formulation from the reservoir into the duodenum during the period of the pump activity. In case the formulation does not reach the duodenum in a suitable rate, the action of the pump will be stopped. Accordingly, sensor 30 may be a chemical sensor, fluid sensor, capacitance sensor, and others. Sensor 30 is in communication with controller 20. Upon sensing contact of the formulation 14 with tissue in the peritoneal cavity, controller 20 stops operation of pump 16.

The following describes examples of preparation of a substance for subcutaneous delivery, the substance containing a high concentration of levodopa and carbidopa.

### Example 1

The substance is in powder form. 3.7185 g powder consists of: 1.48 g Levodopa and 0.370 g Carbidopa, which means a total of 1.85 g API (active pharmaceutical ingredient) =47% of the powder is the API. The remainder of the powder consists of 1.85 g random methyl-β-cyclodextrin and 0.0185 g anti-oxidant. Examples of anti-oxidants include, without limitation, ascorbic acid, ascorbyl palmitate, and tocopherol succinate.

The powder underwent dissolution in a large volume of acidic water. Examples of acidic water include, without limitation, solutions of phosphoric acid (H₃PO₄) or hydrochloric acid (HCl).

The solution then underwent vortexing and sonication, followed by freezing and lyophilization. It is noted that the invention includes just sonication, just vortexing or a combination of both. The sonication may be done, without limitation, ultrasonically at 40 KHz. Alternatively, instead of lyophilization, the substance may undergo grinding or milling, such as jet milling. Afterwards, the substance was reconstituted in a 1 g reconstitution matrix to arrive at the final formulation. The matrix was selected from acidic water (pH=1.5), polyethylene glycol 400 or polyvinylpyrrolidone (PVP).

$$\begin{matrix}\text{The} \text{resultant} \text{weight} \text{of} \text{the} \text{solution} = \\ \text{3} {\text{.7185}}_{\text{powder}} {\text{+1}}_{\text{matrix}} \text{=} 4.7185 \text{g}\end{matrix}$$

$$\begin{matrix}\% \text{API} {}_{\text{w} / \text{w}} \text{=} \\ \text{1} {\text{.85}}_{\text{APIweight}} / \left(\text{3} {\text{.7185}}_{\text{powder} \text{weight}} {\text{+1}}_{\text{matrix} \text{weight}}\right) \times \\ 100 = 39.21 \% \text{w} / \text{w}\end{matrix}$$

$$\begin{matrix}\% {\text{API}}_{\text{w} / \text{v}} = \text{\%API} {}_{\text{w} / \text{w}} \text{X} \text{sample} \text{density} \text{=39} \text{.21} \times \text{1} \text{.2} = \\ \text{47\%} {}_{\text{w} / \text{v}} \text{=} 470.5 \text{mg} / \text{ml}\end{matrix}$$

$$\text{Sample} \text{density} = \text{1} \text{.2g} / \text{ml}$$

### Example 2

3.5 ml of 0.2 N HCl were added to 200 mg of L- DOPA, 50 mg of carbidopa and 2.5 mg of ascorbic acid and the mixture was vortexed and then sonicated. Under these conditions L- DOPA did not fully dissolve. Then 1N HCl was added dropwise till all L-DOPA was dissolved.

The procedure was repeated for a mixture of 200 mg of L- DOPA, 50 mg of carbidopa, 2.5 mg of ascorbic acid and 250 mg of hydroxypropyl-beta-cyclodextrin (HPβCD).

Each of the above solutions was lyophilized and reconstituted in 0.4 ml H₂O to yield 0.5 ml solution. Each of the solutions was sonicated. Only the formulation that contained HPβCD was clear, while the formulation without HPβCD was very turbid as the L-DOPA/carbidopa did not dissolve.

The formulation that contained HPβCD contained 50% of the API weight per volume. The color of this clear solution of the formulation that contained HPβCD was pale yellowish and did not change under storage for at least two weeks.

What is claimed is:

1. A method of treating a neurological disease, comprising:
using a subcutaneously-implanted pump to deliver to a portion of a gastrointestinal tract a formulation comprising a levodopa compound and a carbidopa compound which provides a treatment for a neurological disease.

2. The method according to claim 1, wherein a viscosity of said formulation is not greater than 50 centipoise at 30-41° C.

3. The method according to claim 1, wherein a viscosity of said formulation is in a range of 1-50 centipoise at 25° C.

4. The method according to claim 1, wherein said formulation also comprises an intestinal absorbance enhancer.

5. The method according to claim 1, wherein a ratio between the levodopa compound and the carbidopa compound is in a range of 3.5:1 - 10:1.

6. The method according to claim 1, wherein said levodopa compound is in a range of 300-1000 mg/ml and said carbidopa compound is in a range of 75-300 mg/ml.

7. The method according to claim 1, wherein said levodopa compound and said carbidopa compound constitute together at least 30% by weight of said formulation.

8. The method according to claim 1, wherein said levodopa compound and said carbidopa compound constitute together 50% by weight of said formulation.

9. The method according to claim 1, wherein said neurological disease is Parkinson’s disease.

10. The method according to claim 1, using a controller in communication with said pump to control operation of said pump, and using a sensor which is in communication with said controller to sense if said formulation contacts tissue in a peritoneal cavity outside a duodenum of said gastrointestinal tract.

11. The method according to claim 10, comprising stopping operation of said pump if said sensor senses that the formulation contacts tissue in the peritoneal cavity outside the duodenum.

12. A method of making a formulation comprising a levodopa compound and a carbidopa compound, comprising:
dissolving a powder comprising a levodopa compound and a carbidopa compound in acidic water to form a solution;
performing sonication or vortexing, freezing, and lyophilization or grinding or milling, of said solution to form a substance; and
performing reconstitution of said substance in a reconstitution matrix to form a formulation, wherein the levodopa compound and the carbidopa compound are at least 30% by weight of said formulation.

13. The method according to claim 12, wherein sonication and vortexing of said solution are done.

14. The method according to claim 12, wherein a viscosity of said formulation is not greater than 50 centipoise at 37° C.

15. The method according to claim 12, wherein a viscosity of said formulation is in a range of 1-50 centipoise at 25° C.

16. The method according to claim 12, wherein a ratio between the levodopa compound and the carbidopa compound is in a range of 3.5:1 - 10: 1.

17. The method according to claim 12, wherein said levodopa compound is in a range of 300-1000 mg/ml and said carbidopa compound is in a range of 75-300 mg/ml.

18. The method according to claim 13, wherein said matrix comprises water, acidic water with a pH in a range of 1-3, polyethylene glycol or polyvinylpyrrolidone.

19. The method according to claim 1, wherein said levodopa compound comprises levodopa, or levodopa salts or levodopa ester including ethyl or propyl isopropyl esters, or other levodopa products, and said carbidopa compound comprises carbidopa, or carbidopa ester including carbidopa propyl ester or carbidopa isopropyl ester.

20. The method according to claim 1, wherein said formulation comprises benserazide or other dopa decarboxylase inhibitor.
